(19) United States
(12) Patent Application Publication
(10) Pub. No.: US 2018/0000514 A1
(43) Pub. Date: Jan. 4, 2018

(54) SHAFT-TYPE INSTRUMENT FOR SURGICAL PURPOSES

(71) Applicant: QENUS MEDICAL AG, Appenzell (CH)

(72) Inventors: Martin KOLLER, Appenzell (CH); Fabrice KÜLLING, Gossau (CH)

(21) Appl. No.: 15/542,741

(22) PCT Filed: Dec. 15, 2015
(86) PCT No.: PCT/EP2015/079822

§ 371 Date: Jul. 11, 2017

(30) Foreign Application Priority Data
Jan. 26, 2015 (EP) 15152482.4

(51) Int. Cl. A61B 17/34; A61B 17/221; A61B 17/16; A61B 17/32; A61B 17/22; A61B 17/00; A61B 17/29

(52) U.S. Cl. CPC A61B 17/3417; A61B 17/1631; A61B 17/221; A61B 2017/2919; A61B 2017/291; A61B 2017/22072; A61B 17/1611; A61B 2017/00367; A61B 2017/00358; A61B 2017/00331; A61B 2017/320024; A61B 2017/320064; A61B 2017/0042

(57) ABSTRACT

The shaft-type instrument (1) has a shaft part (2), fixedly connected at one end to a shaft grip (3), and a sliding part (4). The sliding part (4) is displaceably mounted relative to the shaft part (2) and is, at one end, in operative connection with a pretensioned handgrip (5) such that by actuating the handgrip (5), the sliding part (4) is displaceable counter to the pretension. A respective lever arm (7, 7′) is articulated at two spaced apart shaft articulation points (6, 6′) on the shaft part (2) or shaft grip (3). The handgrip (5) has a coupling section (8) which connects the lever arms (7, 7′) and coupling joints (9, 9′) to one another such that same can pivot in the same direction. The coupling joints are spaced apart and a grip section (10) extends transverse to the coupling section (8) and to the shaft part (2).

The invention relates to a shaft-type instrument for surgical purposes according to the preamble of claim 1. Instruments of this kind are used, among other things, for performing resections on body areas that are in part difficult to access or for removing tissue samples, for example in a laminectomy procedure.

Comparable instruments of the kind in question have been known and customary in surgery for many years. Thus, EP 1 491 155 A1 describes a bone and tissue punch designed as a sliding shaft instrument. Said instrument has an elongate shaft, which is fixedly connected at one end to a shaft grip. A slide is mounted on the shaft in such as way as to be displaceable in the longitudinal direction. The slide is operatively connected to a spring-pretensioned slide grip and can be displaced by actuation of the slide grip. The force required for this purpose and applied by the surgeon's hand is taken up by the slide grip via a rotation movement.

A disadvantage of the known shaft-type instruments is that, when they are actuated, it is easy for the instrument to tilt in the operator's hand. This makes it difficult in particular to precisely guide the tip of the shaft during the actuation of the instrument. It is therefore an object of the invention to overcome the disadvantages of the prior art.

It is in particular an object of the invention to make available a shaft-type instrument of the type mentioned at the outset, which instrument is versatile in use and is of a simple design and can be guided and actuated with a high degree of precision and safety. In addition, the instrument should have a compact structure and a high degree of robustness. Moreover, it should be able to be handled efficiently in a clinical environment, in particular being easily sterilizable. Furthermore, the shaft-type instrument should be as durable as possible and should be able to be manufactured at a favorable price.

These objects are achieved by a shaft-type instrument having the features of claim 1.

The invention relates to a shaft-type instrument for surgical purposes, with a shaft part, which is fixedly connected at one end to a shaft grip. The shaft-type instrument moreover has a sliding part, which is mounted so as to be displaceable relative to the shaft part in the longitudinal direction thereof. The sliding part is operatively connected at one end to a pretensioned handgrip in such a way that, by actuation of the handgrip, the sliding part is displaceable counter to the pretensioning. The invention is characterized in that a respective lever arm is articulated on the shaft part or on the shaft grip at at least two shaft joints spaced apart from each other. Moreover, the handgrip has a coupling portion which connects the at least two lever arms to each other at spaced apart coupling joints so as to be pivotable in the same direction, and a grip portion which extends transversely with respect to the coupling portion and to the shaft part.

By the suspension of the handgrip on several lever arms, the force transmission by hand to the grip portion corresponds more to a translation than a rotation. This permits a better transmission of force to the instrument, but especially a more stable guiding of the tip of the shaft.

The force transmission is improved in particular by the fact that each finger acting on the handgrip transmits its force to the lever arms via the coupling joints. A greater leverage is thereby achieved.

It goes without saying that the present grip structure can be used in shaft-type instruments of all kinds, particularly in sliding shaft instruments and tubular shaft instruments. Thus, said structure can be used not only in bone and tissue punches but also in scissor instruments or grip instruments.

The coupling joints can lie on the ends of the lever arms opposite of the shaft part or shaft grip. This articulation of the lever arms by the handgrip achieves a maximum leverage in a shaft-type instrument according to the invention.

However, it is also possible that the coupling joints are arranged in a central area of at least one lever arm. This allows further elements, e.g. locking means, to be mounted on the lever arms.

At least one lever arm can be designed as a double lever arm with a force arm and a load arm, wherein the force arm is operatively connected to the coupling portion of the handgrip, and the load arm is operatively connected to the sliding part. This structure permits a particularly effective mechanical coupling of the lever arm to the sliding part along with a compact configuration of the shaft-type instrument.

The shaft joints can be offset in the transverse direction with respect to the shaft part. An offset arrangement of the shaft joints on the shaft part or on the shaft grip is advantageous in particular when a lever arm is designed as a double lever arm, since a much more compact structure of the instrument is then permitted.

The ratio of the length of the longest lever arm to the longest distance between two coupling joints in the rest position can be in a range of 2 to 20, preferably 3 to 10, more preferably 4 to 6. This dimensioning of the grip structure achieves the best possible force transmission.

A stop element can be mounted on at least one of the lever arms and defines the angle position of the lever arm in the rest position. This stop element can be steplessly adjustable. Before using the shaft-type instrument, it is thus possible for the surgeon to set the rest position thereof and thereby adapt it to the intervention that is to be performed.

The sliding part is designed as a slide arranged parallel to and next to the shaft part. Such shaft-type instruments are also referred to as sliding shaft instruments. A sliding shaft instrument has the advantage that the slide can be easily separated from the shaft section to permit cleaning, in particular sterilization, before a surgical intervention. The surfaces lying on the inside are in this way also easily accessible. The slide can thus be articulated on the shaft part in such a way that it can be freed from the shaft part by a swivel movement. This permits cleaning of the instrument without the slide and shaft part having to be separated. This simplifies handling in everyday clinical practice and avoids a situation where two elements of different units are joined together after the cleaning procedure.

In an embodiment not according to the invention, the shaft part and the sliding part can also be configured as two substantially coaxially arranged elements, wherein the shaft part forms a preferably tubular receiving element for the sliding part. This structure of a shaft-type instrument is also referred to as a tubular shaft instrument. Tubular shaft instruments have the advantage of having no parts displaceable relative to each other on their outer surface. This particularly facilitates their actuation in especially narrow access channels to an operating site.

The ratio of the length of the shaft to the length of the longest lever arm can be in a range of 1 to 10, preferably 1.2 to 4, more preferably 1.5 to 2.8. Such a shaft-type instrument has particularly advantageous ergonomics and can be easily guided in a surgeon's hand. Moreover, this ratio also ensures an acceptable deflection of the tip of the shaft upon complete actuation of the handgrip.

The shaft part and the sliding part can be connected releasably to the shaft grip, preferably via a reversible form-fit connection, in particular via a dovetail with a locking pin. By virtue of the fact that the shaft part and the sliding part are separable from the shaft grip, different shaft configurations can be mounted on one and the same shaft grip. It is thus conceivable to make available a system of shaft-type instruments in which various shaft configurations with different shaft lengths or with different tools can be mounted on a shaft grip of identical configuration. By virtue of this modular configuration, a large number of different shaft instruments for different areas of use can easily be made available.

The pretensioning can be generated by a spring element mounted on the shaft grip and acting on one of the lever arms, in particular by a leaf spring. It is thus possible to reliably ensure, with a particularly simple structure, that the handgrip adopts its rest position again after actuation of the shaft-type instrument. For this purpose, one of the lever arms can have an anchoring structure for the spring element, in particular a guide groove for a leaf spring. Moreover, the spring element can have a sliding body anchored in the anchoring structure, in particular a ball mounted on a leaf spring. This permits reliable guiding of the spring element.

The shaft part can have a guide profile and the slide can have an engagement element engaging therein. However, it is also possible that the slide has a guide profile and the shaft part has an engagement element engaging therein. The combination of guide profile and engagement element allows the slide to be mounted on the shaft in such a way as to be particularly stable thereon and displaceable in the longitudinal direction. It goes without saying that slide and shaft part can also have several guide profiles or engagement elements. The guide profile and the engagement element can each be formed in one piece with the shaft and the slide. Such a shaft-type instrument has a smaller number of individual parts and is easy to clean and sterilize.

The stop element, in a so-called locking position, can define the angle position of the lever arm in the rest position. In addition, it can be movable, in particular displaceable or pivotable, to a so-called unlocking position. With the stop element in the unlocking position, the lever arm can be movable beyond the rest position counter to the direction of actuation, and the slide can thereby be at least partially releasable from the shaft part. In this configuration, the slide can be releasable from the shaft part by withdrawal of at least one engagement element from a guide profile counter to the direction of actuation. This represents a particularly simple solution for reliably securing the shaft-type instrument in its assembled state and for easily dismantling it after unlocking.

At the end opposite the force arm, the load arm can have a fork profile into which a bolt mounted on the sliding part can be inserted. This permits particularly reliable transmission of force from the force arm to the sliding part, wherein the shaft-type instrument can still be easily dismantled since the bolt can be easily withdrawn from the fork profile.

The fork profile can have an asymmetrical configuration, wherein the side of the fork profile acted on by the bolt mounted on the sliding part, upon actuation of the shaft-type instrument, has a greater material thickness. Experiments have shown that, with a symmetrical fork profile, there is a danger of the latter being deformed under the forces that arise during the use of the shaft-type instrument. This danger can be counteracted by the greater material thickness of the fork profile on the side that is subjected to a greater load.

Further advantages and individual features of the invention will become clear from the following description of an illustrative embodiment and from the schematic drawings, in which:

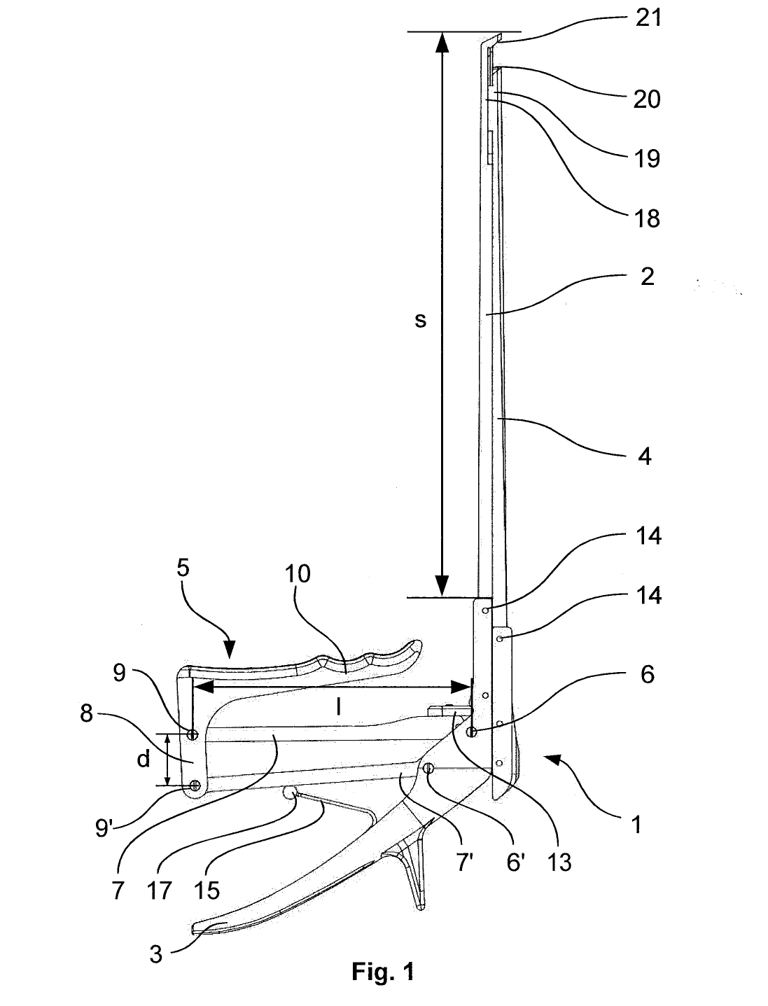

FIG. 1 shows a side view of a shaft-type instrument according to the invention in an opened state;

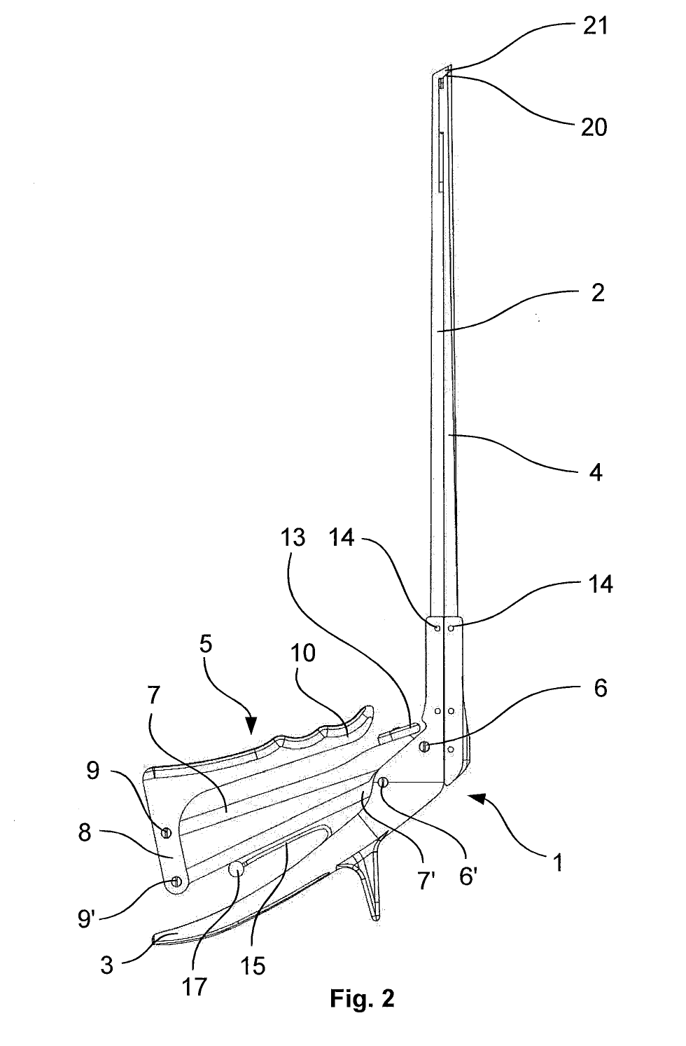

FIG. 2 shows the shaft-type instrument according to the invention from FIG. 1 in a closed state;

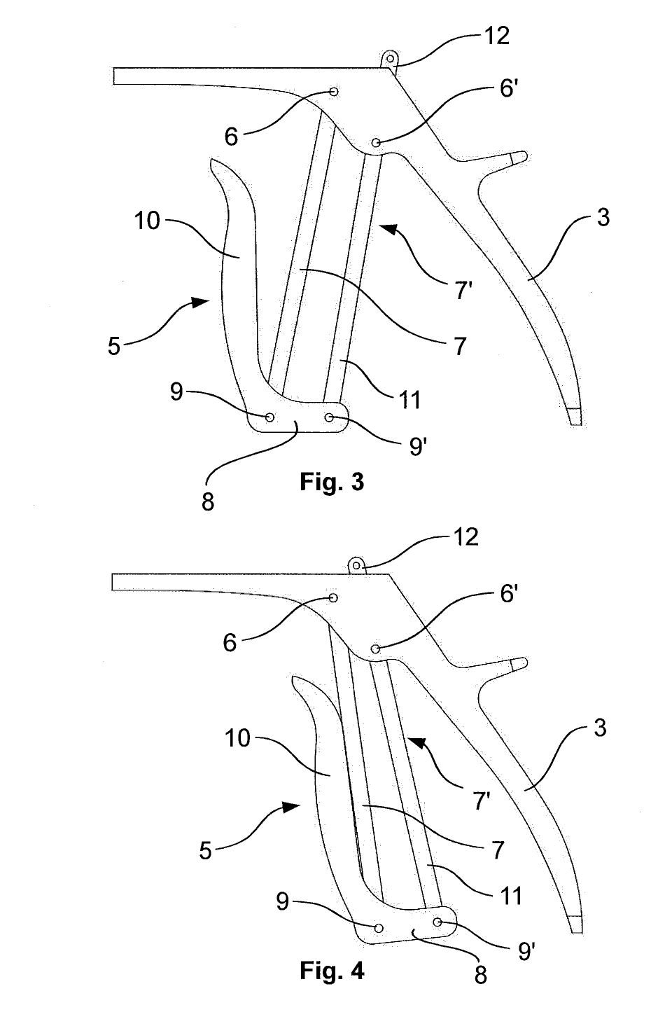

FIG. 3 shows a diagrammatic side view of the grip structure of a shaft-type instrument according to the invention in the opened state;

FIG. 4 shows a diagrammatic view as per FIG. 3 in closed state;

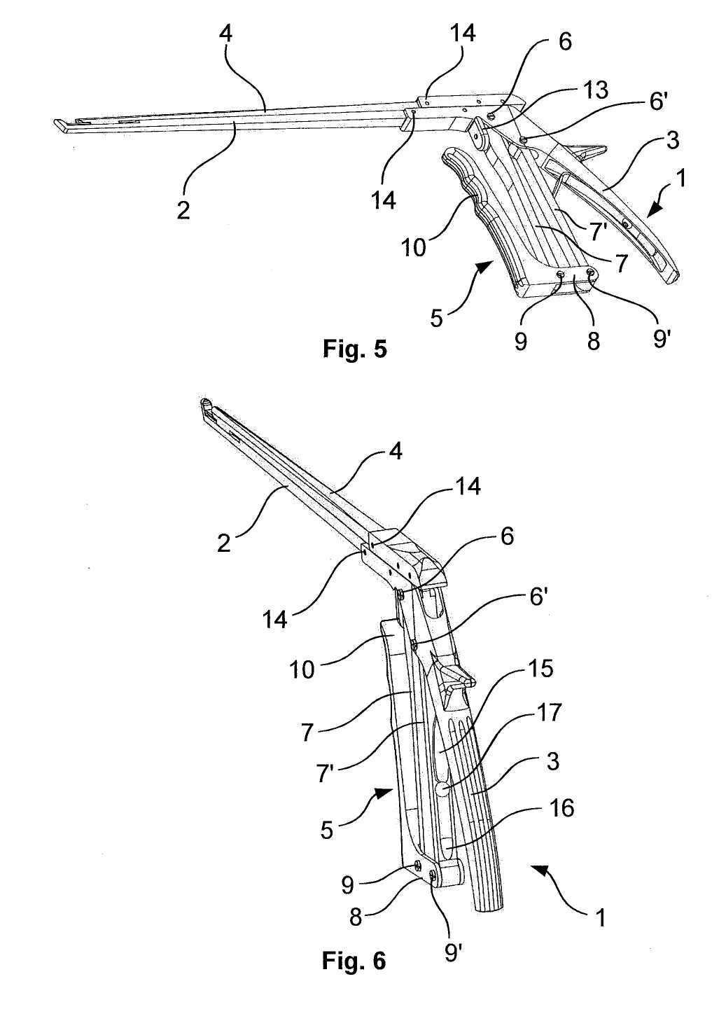

FIG. 5 shows a perspective view of a shaft-type instrument according to the invention from FIGS. 1 and 2;

FIG. 6 shows a perspective view of the shaft-type instrument according to the invention from FIG. 5;

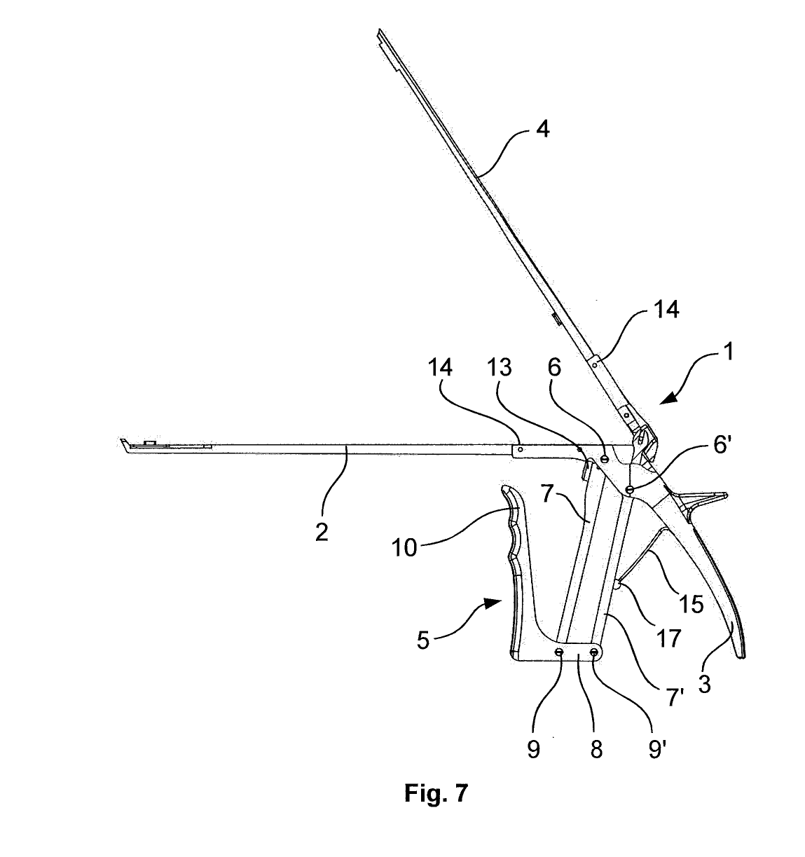

FIG. 7 shows a side view of the shaft-type instrument according to the invention from FIG. 6 in a swiveled-open state;

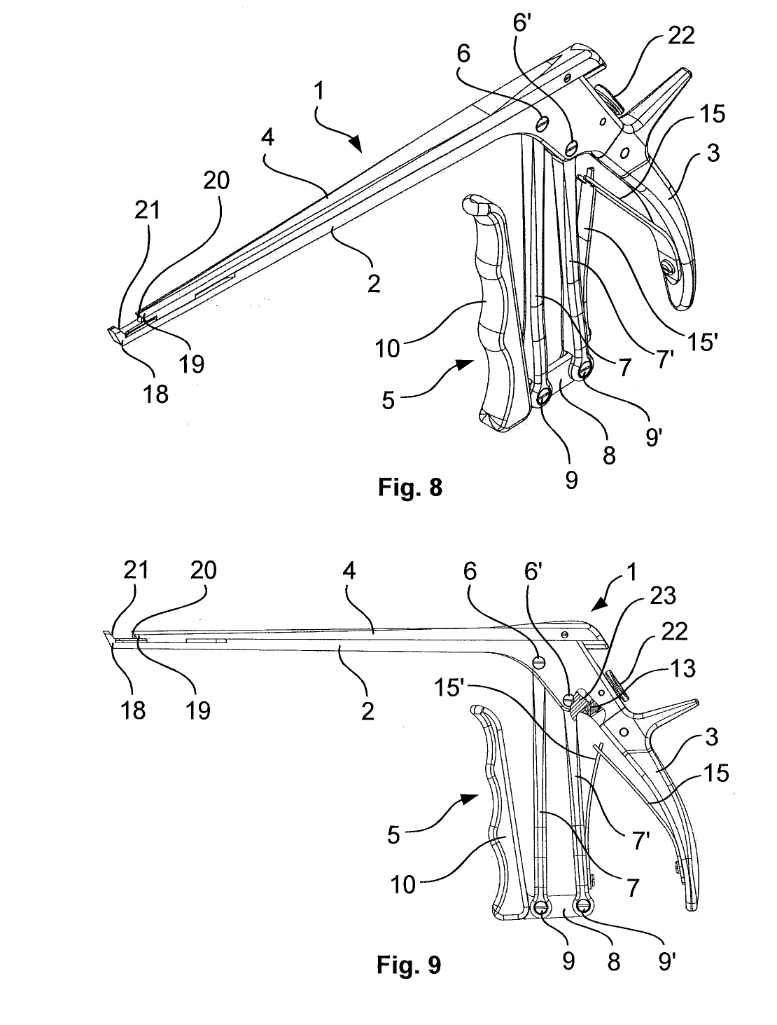

FIG. 8 shows a perspective view of a further illustrative embodiment of a shaft-type instrument according to the invention;

FIG. 9 shows a side view of a shaft-type instrument from FIG. 8, partially in cross section;

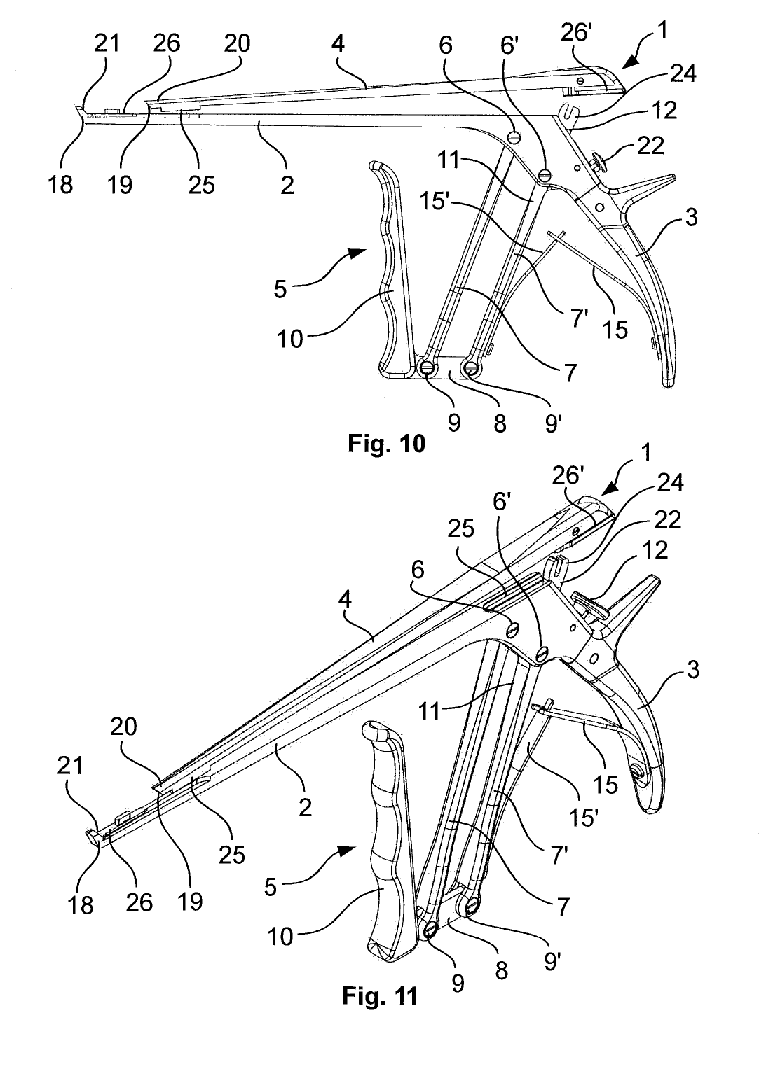

FIG. 10 shows a side view of a shaft-type instrument from FIGS. 8 and 9 in a dismantled state;

FIG. 11 shows a perspective view of a shaft-type instrument from FIGS. 8 to 10 in a dismantled state.

FIG. 1 shows a side view of a shaft-type instrument 1 according to the invention in an opened state. Said instrument 1 has a shaft part 2, which is anchored on the shaft grip 3. Moreover, a sliding part (or slide) 4 is arranged parallel to the shaft part 2. In the present case, the instrument 1 is a laminectomy punch in which a punch abutment 21 is arranged at the tool end 18 of the shaft part 2, and a punching portion 20 is arranged at the tool end 19 of the slide 4. However, the nature and configuration of the tool can vary depending on the intended use, and it would also be conceivable, for example, for a scissor-type cutting tool to be actuated via the slide 4.

It will also be seen that the lever arms 7 and 7′ are articulated on the shaft grip 3 via the shaft joints 6 and 6′. At their ends directed away from the shaft grip 3, the lever arms 7 and 7′ are connected to the coupling portion 8 of the handgrip 5 via the coupling joints 9 and 9′. The handgrip 5 moreover has a grip portion 10 for its actuation.

In the shaft-type instrument shown, the shaft part 2 has the length s. The length of the longer lever arm 7 is given by the extent 1, and the spacing between the two coupling joints 9 and 9′ in the rest position is given by the distance d.

FIG. 2 shows the shaft-type instrument according to the invention from FIG. 1 in a closed state. It will be seen that, by actuation of the handle 5, the punching portion 20 is pressed against the punch abutment 21. In the course of this movement, the slide 4 is displaced in the longitudinal direction relative to the shaft part 2 counter to the pretensioning of the leaf spring 15.

The grip configuration of a shaft-type instrument 1 according to the invention in the opened state is shown in detail in FIG. 3. It will be seen that the lever arm 7′ is designed as a double lever arm with a force arm 11 and a load arm 12. The end of the load arm 12 is designed such that it can act on the sliding part 4.

The grip configuration according to FIG. 3 is shown in the closed state in FIG. 4. In the illustrative embodiment shown, the movement of the lever arm 7 from the opened state to the closed state leads to a rotation about an angle of 25°. By contrast, the same movement leads to a rotation of the handgrip 5 about only 10°.

FIG. 5 shows a perspective view of a shaft-type instrument according to the invention from FIGS. 1 and 2. It will be seen that a stop element 13, which defines the rest position of the shaft-type instrument 1, is mounted on the lever arm 7. In the shaft-type instrument shown, the shaft part 2 and the sliding part 4 are each connected releasably to the shaft grip 3 via a reversible form-fit connection 14, in particular via a dovetail with locking pin. In addition, the spring element for pretensioning the handgrip 5 is designed as a leaf spring 15.

FIG. 6 shows another perspective view of a shaft-type instrument 1 according to the invention from FIGS. 1 and 2. The pretensioning mechanism is shown more clearly here. In particular, a guide groove 16 for the leaf spring 15 can be seen on the lever arm 7′. A ball 17 is mounted as a sliding body on the leaf spring 15 and runs in the guide groove 16.

FIG. 7 shows a side view of a shaft-type instrument according to the invention from FIG. 6. Here, the sliding part or slide 4 is swiveled away from the shaft part 2 in order to free the two parts from each other, for example for cleaning purposes. In this position, the instrument 1 can be placed in a sterilizing basket with other instruments, said instrument 1 remaining at all times connected as one unit.

FIGS. 8 to 11 show a further illustrative embodiment of a shaft-type instrument 1 according to the invention. On the side facing the user during use, said instrument 1 has a button 22 whose actuation allows the shaft-type instrument 1 to be dismantled. The button 22 is connected to the stop element 13 and is mounted with spring pretensioning. In the assembled state, the stop element 13 defines, together with the stop 23 on the lever arm 7′, the angle position thereof in the rest position.

To dismantle the shaft-type instrument 1, the user has to press the handgrip 5 and the grip portion 10 at least slightly together, so that the stop 23 lifts away from the stop element 13. Thereafter, the button 22 can be lifted counter to the spring pretensioning and rotated through 90°, as a result of which the stop element 13 is removed from the stop 23 and is locked in this position. When the handgrip 5 and the grip portion 10 are then let go, the lever arm 7′ moves beyond the rest position counter to the direction of actuation. In this way, the engagement elements 26 and 26′ on the slide 4 and on the shaft part 2, respectively, are withdrawn from the guide profiles 25 and 25′, as a result of which the slide 4 can be removed completely from the shaft part 2 (cf. FIGS. 10 and 11). The shaft-type instrument 1 is assembled in the reverse sequence.

As will be seen from FIGS. 10 and 11, the end of the load arm 12 opposite of the force arm 11 is designed as a fork profile 24. In this way, during dismantling, the slide 4 can be separated in a particularly simple manner from the rest of the shaft-type instrument. The fork profile 24 has an asymmetrical configuration. The side of the fork profile 24 acted on by the slide 4 has a greater material thickness. It is thereby possible to avoid a deformation of the fork profile 24 as a result of the forces that arise during use of the shaft-type instrument 1.

1-15. (canceled)

16. A shaft-type instrument for surgical purposes, with a shaft part, which is fixedly connected at one end to a shaft grip, and with a sliding part, which is mounted so as to be displaceable relative to the shaft part in a longitudinal direction thereof and is operatively connected, at one end, to a pretensioned handgrip in such a way that, by actuation of the handgrip, the sliding part is displaceable counter to the pretensioning, and the sliding part being designed as a slide arranged parallel to and adjacent the shaft part,
wherein a respective lever arm is articulated on the shaft part or on the shaft grip at least two shaft joints spaced apart from each other,
the handgrip has a coupling portion which connects the at least two lever arms to each other at spaced apart coupling joints so as to be pivotable in the same direction, and
a grip portion extends transversely with respect to the coupling portion and to the shaft part.

17. The shaft-type instrument according to claim 16, wherein the coupling joints lie at those ends of the lever arms opposite of the shaft part or shaft grip.

18. The shaft-type instrument according to claim 16, wherein at least one lever arm is designed as a double lever arm with a force arm and a load arm,
the force arm is operatively connected to the coupling portion of the handgrip, and
the load arm is operatively connected to the sliding part.

19. The shaft-type instrument according to claim 16, wherein the shaft joints are offset, in a transverse direction, with respect to the shaft part.

20. The shaft-type instrument according to claim 16, wherein a ratio of a length of a longest lever arm to a longest distance between two coupling joints, in a rest position, is in a range of 2 to 20.

21. The shaft-type instrument according to claim 20, wherein, the ratio of the length of the longest lever arm to the longest distance between two coupling joints, in the rest position; is in a range of 3 to 10.

22. The shaft-type instrument according to claim 20, wherein, the ratio of the length of the longest lever arm to the longest distance between two coupling joints, in the rest position, is in a range of 4 to 6.

23. The shaft-type instrument according to claim 16, wherein a stop element is mounted on at least one of the lever arms and defines the angle position of the lever arm in the rest position.

24. The shaft-type instrument according to claim 16, wherein the slide is at least partially releasably connected to the shaft part.

25. The shaft-type instrument according to claim 24, wherein the slide is articulated on the shaft part in such a way that the slide can be freed from the shaft part by a swivel movement.

26. The shaft-type instrument according to claim 16, wherein a ratio of a length of the shaft to a length of a longest lever arm is between 1 and 10.

27. The shaft-type instrument according to claim 26, wherein the ratio of the length of the shaft to the length of the longest lever arm is between 1.2 and 4.

28. The shaft-type instrument according to claim 27, wherein the ratio of the length of the shaft to the length of the longest lever arm is between 1.5 and 2.8.

29. The shaft-type instrument according to claim 16, wherein the pretensioning is generated by a spring element mounted on the shaft grip and acting on one of the lever arms.

30. The shaft-type instrument according to claim 16, wherein the shaft part has a guide profile and the slide has an engagement element engaging therein, in that the slide has a guide profile and the shaft part has an engagement element engaging therein.

31. The shaft-type instrument according to claim 21, wherein the stop element, in a locking position, defines the angle position of the lever arm in the rest position and is additionally movable to an unlocking position, and, with the stop element in the unlocking position, the lever arm is movable beyond the rest position counter to a direction of actuation, and the slide is thereby at least partially releasable from the shaft part.

32. The shaft-type instrument according to claim 24, wherein the stop element, in a locking position, defines the angle position of the lever arm in the rest position and is additionally movable to an unlocking position, and, with the stop element in the unlocking position, the lever arm is movable beyond the rest position counter to a direction of actuation, and the slide is thereby at least partially releasable from the shaft part.

33. The shaft-type instrument according to claim 30, wherein the slide is releasable from the shaft part by withdrawal of at least one engagement element from a guide profile counter to a direction of actuation.

34. The shaft-type instrument according to claim 31, wherein the slide is releasable from the shaft part by withdrawal of at least one engagement element from a guide profile counter to a direction of actuation.

35. The shaft-type instrument according to claim 32, wherein the slide is releasable from the shaft part by withdrawal of at least one engagement element from a guide profile counter to a direction of actuation.

36. The shaft-type instrument according to claim 18, wherein the load arm has a fork profile, at an end opposite the force arm, into which a bolt mounted on the sliding part can be inserted.

37. The shaft-type instrument according to claim 36, wherein the fork profile has an asymmetrical configuration, the side of the fork profile acted on by the bolt mounted on the sliding part, upon actuation of the shaft-type instrument, has a greater material thickness.
